(19) United States
(12) Patent Application Publication
(10) Pub. No.: US 2021/0030575 A1
(43) Pub. Date: Feb. 4, 2021

(54) JOINT STABILIZER FOR ADAPTIVE DAMPING OF A MOVEMENT OF A BODY

(71) Applicant: BETTERGUARDS TECHNOLOGY GmbH, Berlin (DE)

(72) Inventors: Vinzenz BICHLER, Berlin (DE); Timo STUMPER, Berlin (DE)

(73) Assignee: BETTERGUARDS TECHNOLOGY GmbH, Berlin (DE)

(21) Appl. No.: 16/321,796

(22) PCT Filed: Jul. 28, 2017
(86) PCT No.: PCT/EP2017/069207

§ 371 Date: Jan. 29, 2019

(30) Foreign Application Priority Data
Jul. 29, 2016 (DE) 102016114110.0

(51) Int. Cl. A61F 5/01

(52) U.S. Cl. CPC A61F 5/013; A61F 2005/0169

(57) ABSTRACT

A body joint stabilizing apparatus for adaptively damping a body movement, comprising: a receptacle, wherein the receptacle is filled with a shear-thickening medium, a tensioning body that is movable relative to the receptacle, wherein the tensioning body is connectable to a body region of the user that is movable relative to another body region of the user, on which the receptacle is fastenable, an effector body that is displaceably arranged in the receptacle and provided for an interaction with the shear-thickening medium, and a connection element for transmitting forces between the receptacle and the tensioning body, wherein the receptacle, the tensioning body and the effector body are connected to one another by way of the connection element, wherein the to connection element is guided via at least one deflection means for the purposes of dividing the force acting on the connection element.

### CROSS REFERENCE TO RELATED APPLICATIONS

The present application is a United States National Stage application of International Patent Application PCT/EP2017/069207, filed on Jul. 28, 2017, which claims foreign priority to German Patent Application No. DE 10 2016 114 110.0 filed on Jul. 29, 2016, the entirety of each of which is incorporated by reference hereby.

### FIELD OF THE RELATED ART

The present invention relates to a body joint stabilizing apparatus for adaptively damping a body movement.

### BACKGROUND

The practice of stabilizing body joints, muscles and tendons by means of apparatuses that facilitate an adaptive movement restriction is known. The adaptive behavior of such apparatuses is achieved, inter alia, by virtue of two bodies moving relative to one another, with a shear-thickening fluid being situated between the bodies. The opposing faces of the bodies form shear surfaces in this case, which introduce shear forces into the shear-thickening fluid on account of the relative movement. The shear-thickening fluid becomes more viscous with increasing shear forces. Above a defined shear speed, the shear-thickening fluid experiences a jump in the shear, as a result of which the degree of viscosity rapidly increases.

The apparatuses are fixed between two body sites on the user. Here, one shear body of the apparatus forms a receptacle, which is filled with the shear-thickening fluid. The other shear body forms a pullout body, which is arranged in the receptacle in movable fashion. If physiological forces, i.e., uncritical forces in relation to the body part to be stabilized in appropriate fashion, are introduced into the apparatus via the two body sites of the user, a relative movement between receptacle and pullout body, and hence the movement of the body part to be stabilized, is admitted on account of the liquid state of the shear-thickening fluid.

By contrast, if non-physiological forces, i.e., critical forces in relation to the body part to be stabilized in appropriate fashion, are introduced into the apparatus, the shear forces emanating from the shear surfaces of the receptacle and of the pullout body cause shear thickening of the shear-thickening fluid, as a result of which relative movement between the pullout body and the receptacle is only still possible with great effort.

Such an apparatus is known from WO 2013/174989 A1, for example, which discloses an orthopedic apparatus for limiting the movement of a joint arranged between a first and a second body region.

The joints in the human body have very different physiological freedoms of movement and can deflect very different forces. By way of example, when bending the knee, the physiological relative movement between thigh and lower leg and the corresponding forces are very much greater than when, for example, bending a finger. According to the prior art, there is a direct (linear) connection between the two connected body parts. As a consequence, very large apparatuses with space for a corresponding large deflection have to be built in the case of very large movements; under certain circumstances, these may be difficult to attach or uncomfortable for the users. By contrast, in the case of very small biomechanical defections, the structure of a correspondingly delicate apparatus is difficult to realize from a technical point of view since the available path for developing the protective effect is comparatively small.

### SUMMARY

Proceeding from the known prior art, it is an object of the present invention to specify a body joint stabilizing apparatus for adaptively damping a body movement, which decouples the absolute magnitude of the relative movement of the body parts from the size of the body joint stabilizing apparatus such that the latter can be designed substantially freely and such that the same apparatuses can be used on different joints as well.

This object is achieved by means of a body joint stabilizing apparatus having the features of claim 1. Advantageous configurations emerge from the dependent claims.

Described is a body joint stabilizing apparatus for adaptively damping a body movement, comprising: a receptacle, wherein the receptacle is filled with a shear-thickening medium, a tensioning body that is movable relative to the receptacle, wherein the tensioning body is connectable to a body region of the user that is movable relative to another body region of the user, on which the receptacle is fastenable, an effector body that is displaceably arranged in the receptacle and provided for an interaction with the shear-thickening medium, and a connection element for transmitting forces between the receptacle and the tensioning body.

Accordingly, a body joint stabilizing apparatus for adaptively damping a body movement is specified, comprising a receptacle, wherein the receptacle is filled with a shear-thickening medium, a tensioning body that is movable relative to the receptacle, wherein the tensioning body is connectable to a body region of the user that is movable relative to another body region of the user, on which the receptacle is fastenable, an effector body that is displaceably arranged in the receptacle and provided for an interaction with the shear-thickening medium, and a connection element for transmitting forces between the receptacle and the tensioning body. According to the invention, the receptacle, the tensioning body and the effector body are connected to one another by way of the connection element, wherein the connection element is guided via at least one deflection means for the purposes of dividing the force acting on the connection element.

The body joint stabilizing apparatus can be attached to the body of the user in such a way that it is able to stabilize a joint of the user by means of the adaptive movement restriction function.

Different force conversion scenarios can be provided depending on the sequence in which the receptacle, the tensioning body and the effector body are connected to one another by means of the connection element. Consequently, depending on the field of application, it is possible to increase or reduce the scope and intensity of a movement emanating from a body part in relation to the scope of the movement and the movement intensity of the effector body. As a result, large body joint stabilizing apparatuses can also be used for damping small relative body movements. Conversely, small body joint stabilizing apparatuses thus can also be used in the case of relatively large relative body movements. Overall, the absolute magnitude of the relative movement of the body parts can be decoupled from the size of the body joint stabilizing apparatus. As a result, the field of application of body joint stabilizing apparatuses can be significantly increased. Moreover, additional degrees of freedom emerge in relation to the design of the body joint stabilizing apparatus, as the latter is influenced less strongly by the field of use of the body joint stabilizing apparatus.

In a preferred embodiment, the connection element has a first end and a second end, wherein the at least one deflection means is guided in movable fashion on the connection element between the first end and the second end. Here, the first end, the second end and the deflection means each form a force application point for transmitting a force between the connection element and the receptacle, the tensioning body and the effector body, wherein the receptacle, the tensioning body and the effector body are each securely connected to a force application point. Here, the assignment of the receptacle, the tensioning body and the effector body to the corresponding force application point depends on the field of use of the body joint stabilizing apparatus and it varies depending on whether the force that is introducible into the body joint stabilizing apparatus via the tensioning body is intended to be increased or reduced.

In a preferred development, the first end of the connection element is connected to the effector body or the tensioning body and the second end of the connection element is connected to the receptacle or the tensioning body.

By way of example, if the connection element is connected to the effector body at the first end and to the receptacle at the second end, with the tensioning body being movably guided on the connection element between the first and second end by means of the deflection means, it is possible for the effector body to travel a path through the shear-thickening medium that is longer than the path by which the tensioning body is deflected as a consequence of an external force. In particular, the ratio of the path of the effector body to the path of the tensioning body thus can be 2:1. Overall, the body joint stabilizing device acts much more sensitively and already reacts to small movements of the tensioning body.

This can ensure that the scope of the movement can be substantially doubled if there is only a minor relative movement between the body region of the user on which the receptacle is fastened and the body region of the user on which the tensioning body is fastened, and it is possible to produce a relative movement that is as large as possible between the effector body and the receptacle. This can ensure that sufficient interaction between the effector body and the shear-thickening medium is caused, even in the case where little movement is introduced into the body joint stabilizing apparatus.

Moreover, the force introduced into the body joint stabilizing apparatus via the tensioning body can be divided into substantially equal portions among the effector body and the receptacle. This permits a smaller structure of the receptacle and of the effector body. In particular, this consequently allows the receptacle to have a flatter embodiment. By way of example, it is possible to choose thinner wall thicknesses for the receptacle.

By way of example, if the tensioning body is pulled with the force of 1000 N, a force of 500 N acts in each case on the effector body and on the receptacle.

Moreover, the speed experienced by the effector body when the tensioning body is deflected is greater than the speed with which the tensioning body is deflected. Here, the magnitude of the speed of the effector body in the case of a deflection of the tensioning body can be substantially twice as large as the magnitude of the speed with which the tensioning body is deflected.

Consequently, a critical speed of the effector body, at which the shear-thickening medium starts to solidify in the region of the shear surfaces of the effector body, can be obtained, even in the case where comparatively slow body movements act on the body joint stabilizing apparatus. The deflection means deflects the connection element coming from the effector body toward the receptacle.

If, alternatively, the connection element is connected to the tensioning body at a first end and connected to the receptacle at a second end, with the effector body being movably guided on the connection element between the first and the second end by means of the deflection means, it is is possible to reduce the path traveled by the effector body in the receptacle in relation to the path that the tensioning body travels on account of an external force acting thereon. In particular, it is possible that the effector body moves with a ratio of 1:2 in relation to the tensioning body as a consequence of the deflection of said tensioning body. That is to say, the effector body only travels half of the path within the receptacle that the tensioning body is moved on account of an external force.

Such a body joint stabilizing apparatus is suitable for body regions of the user that admit large relative movements. Reducing the path traveled by the effector body within the receptacle facilitates the use of a receptacle with comparatively small dimensions despite a large movement range of the body regions of the user that are movable relative to one another.

Moreover, the deflection means causes the force acting on the effector body to be substantially twice as large as the force acting on the tensioning body.

Further, the speed of the effector body can be reduced in relation to the speed with which the tensioning body and the receptacle move relative to one another. In particular, the speed with which the effector body moves can be half as fast as the relative speed between tensioning body and receptacle. As a result, it is possible, for example, to admit fast physiological movements between two body regions that are stabilized by means that the body joint stabilizing apparatus. As a result, the use of smaller receptacles is possible. It is sufficient to dimension the receptacle and the effector body for speeds that are substantially half as fast as the relative speed between tensioning body and receptacle.

In a preferred embodiment, the deflection means deflects the connection element at least once through an angle of between 150° and 190°. In a further preferred configuration, the deflection means deflects the connection element at least once through substantially 180°. As a result, the deflection means has the function of a force converter. Thus, the force at the two ends of the connection means can be only half as large in each case as the force acting on the body on which the deflection means is fastened.

By way of example, the deflection means can be embodied in the form of a deflection wedge. Here, the deflection wedge can have a circumferential flute or groove, through which the connection element can slide.

In a preferred embodiment, the tensioning body is arranged outside of the receptacle. Accordingly, the tensioning body can be connected to a body part and the receptacle can be connected to another body part, which is movable relative to the one body part. Moreover, the receptacle has at least one opening, through which the connection element leads from the interior of the receptacle to the outside, toward the tensioning body.

In one development, the deflection means has material with a low coefficient of friction. As a result, it is possible to keep the resistance that can be traced back to the dynamic friction between the connection element and the deflection means small. As a result, the frictional force can be virtually neglected when setting up a force equilibrium via the first end of the connection element, the second end of the connection element and the part of the connection element in contact with the deflection means. This can be promoted further by virtue of the material of the connection element also having a low coefficient of friction.

In a further preferred embodiment, the deflection means comprises at least one deflection pulley. The deflection pulley can be a force converter, which may comprise a wheel or a circular disk mounted on a shaft. The deflection pulley allows the connection element to be guided and deflected with little friction.

In a further preferred development, the connection element has a tension-resistant embodiment. As a result, a tensile force can be transmitted in each case the first end of the connection element and the second end of the connection element when a tensile force is introduced into the connection element via the deflection means. As a result, the body joint stabilizing apparatus is suitable for tensile loads, in which the effector body is deflected by way of a tensile force.

In a further preferred configuration, the connection element is embodied in the form of an elongate, flexible tension element. As a result, the connection element is able to follow the geometry of the deflection means.

In a further preferred configuration, the connection element has fibers, a strap, a belt, a rope, a cable and/or a wire. As a result, it is possible to transmit strong forces between the effector body, the tensioning body and the receptacle, and at the same time provide a flexibility of the connection element.

In one development, the connection element has an integral embodiment with the effector body. In so doing, the part of the connection element that is in contact with the shear-thickening medium can form the effector body. In so doing, the part of the surface of the connection element that is in contact with the shear-thickening medium forms the shear surface which introduces shear forces into the shear-thickening medium in the case of a relative movement between the receptacle and the connection element.

With its diameter, the elongate connection element determines the dimensions of the receptacle. As a result, it is possible to build receptacles, and hence body joint stabilizing devices, with particularly small dimensions.

In a preferred configuration, the deflection means comprises at least two deflection pulleys for deflecting the connection element, with one deflection pulley being arranged on the tensioning body and the other deflection pulley being arranged on the effector body or on the receptacle. Depending on whether the second end of the connection element is fastened to the effector body or to the tensioning body, the path along which the effector body is moved on account of the deflection of the tensioning body can be shortened or lengthened by a factor of 1.5.

If the connection element is guided from the receptacle via a deflection pulley on the effector body, via a deflection pulley on the tensioning body and finally to the effector body and fastened on the latter, the deflection of the effector body that can be traced back to a relative movement between the tensioning body and the receptacle can be reduced in relation to this relative movement.

If the connection element is fastened to the first end of the receptacle, runs via a deflection pulley arranged on the tensioning body, runs onward via a deflection pulley arranged on the effector body and runs toward the tensioning body, with the first end of the connection element being fastened to the latter, a deflection of the effector body that can be traced back to a relative movement between the tensioning body and the receptacle can be increased in relation to said relative movement.

In one development, the interaction between the effector body and the shear-thickening medium is configured in such a way that the effector body is movable through the shear-thickening medium if a force acts on the effector body with a speed below a threshold and that the shear-thickening medium inhibits the movement of the effector body if a force acts on the effector body with a speed that is greater than or equal to a threshold.

In a further preferred configuration, the shear-thickening medium comprises solids, in particular polymers or powders and/or a fluid, in particular a paste or a gel, or a combination thereof.

In general, and in the present application in particular, shear-thickening media should be understood to mean copolymer dispersions as disclosed in DE 30 25 562 A1, DE 34 33 085 A1 and DE 39 17 456 A1, for example. The dispersions are composed of emulsion copolymers and metal salts, for example. By way of example, the emulsion copolymers can be polymerized from 1 to 10% by weight of monoolefinicially unsaturated monocarboxylic and/or dicarboxylic acids such as acrylic acid, methacrylic acid, maleic acid and/or fumaric acid, 99 to 90% by weight of other olefinically unsaturated monomers such as styrene, C1-C6 alkyl acrylates such as methyl methacrylate, and 5 to 30% by weight of a carboxylic allyl ester monomer with two or more copolymerizable double bonds, such as diallyl phthalate.

As a rule, 0.1 to 30% by weight, in relation to the copolymers, of metal oxides, metal hydroxides, metal halides, metal carbonates, metal hydrogen sulfates, metal sulfates, and/or metal phosphates are added as metal salts. Furthermore, the shear-thickening liquids contain thinners such as alcohols, glycols, diglycols and trigylcols, formamides and/or water. Reference is made to DE 30 25 562 A1, DE 39 17 456 A1 and EP 1 443 097 A1 for a more detailed composition of the shear-thickening fluid. Moreover, shear-thickening fluids can also be simple dispersions which have shear-thickening properties above a certain solid component.

In a preferred development, the receptacle has an integral embodiment with a frame for fastening the body joint stabilizing apparatus to a body part. The frame may have loops which can be pulled over a body part—an arm, for example. Alternatively, the frame may also have flanges which are fastenable to the body of the user by means of a tape and/or bandaging material. Further, the frame may also be stuck onto a body part. Moreover, the frame may also have tabs that can be placed around a body part of the user and that are affixable by means of a hook-and-loop fastener.

The receptacle in the form of a chamber is integrated into the frame. Moreover, the tensioning body, too, may have a frame in order to fasten the tensioning body to the body of the user.

### BRIEF DESCRIPTION OF THE FIGURES

Preferred further embodiments and aspects of the present invention are explained in more detail by the following description of the figures. In the figures:

FIGS. 1A, 1B and 1C schematically show different views of a body joint stabilizing apparatus for adaptively damping a body movement, by means of which an increase in the movement of the effector body is possible,

FIGS. 2A, 2B and 2C schematically show different views of a body joint stabilizing apparatus for adaptively damping a body movement, by means of which a reduction in the movement of the effector body is possible,

FIGS. 3A, 3B and 3C schematically show different views of a body joint stabilizing apparatus for adaptively damping a body movement, by means of which an increase in the movement of the effector body is possible,

FIGS. 4A, 4B and 4C schematically show different views of a body joint stabilizing apparatus for adaptively damping a body movement, by means of which a reduction in the movement of the effector body is possible,

FIGS. 5A, 5B and 5C schematically show different views of a body joint stabilizing apparatus for adaptively damping a body movement, by means of which a reduction in the movement of the effector body is possible,

FIGS. 6A and 6B schematically show different perspective views of a body joint stabilizing apparatus for adaptively damping the movement of a wrist,

FIG. 6C shows a detailed view of the body joint stabilizing apparatus of FIG. 6A,

FIG. 6D shows a sectional view of the body joint stabilizing apparatus along the section line H-H of FIG. 6A,

FIGS. 7A, 7B and 7C schematically show different views of a deflection wedge, and

FIGS. 8A, 8B and 8C schematically show different views of a deflection pulley.

### DETAILED DESCRIPTION OF EXEMPLARY EMBODIMENTS

Preferred exemplary embodiments are described below on the basis of the figures. Here, the same or similar elements, or elements with the same effect, are denoted by identical reference signs. In order to avoid redundancy, a repeated description of these elements is partially dispensed with in the following description.

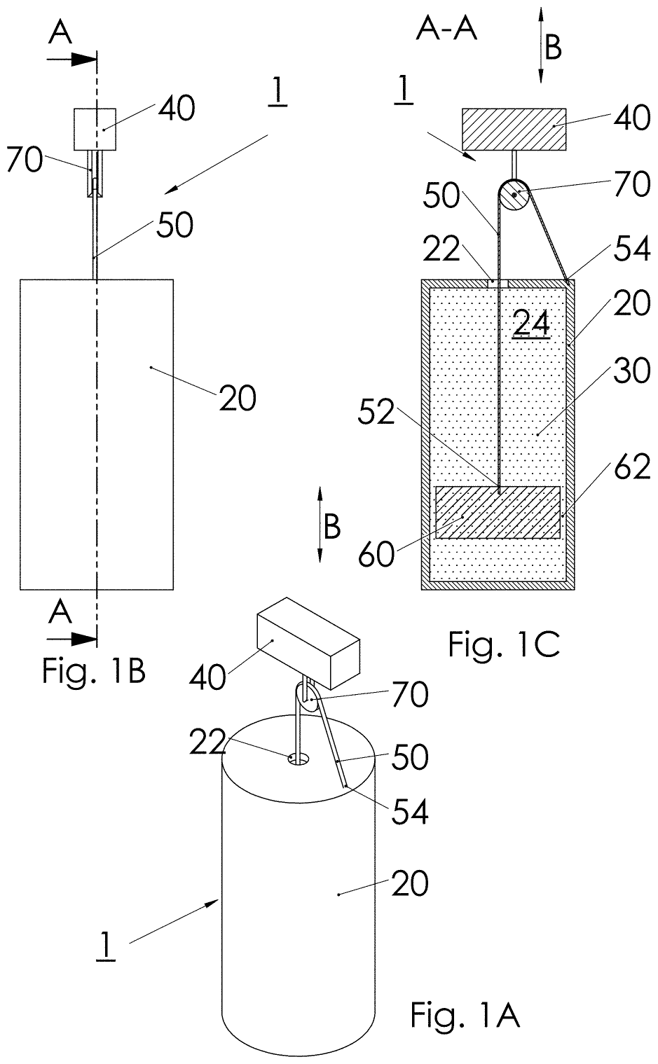

A body joint stabilizing apparatus 1 for adaptively damping a body movement can be gathered from FIGS. 1A, 1B and 1C. The body joint stabilizing apparatus 1 has a cylindrical receptacle 20 for receiving a shear-thickening medium and an effector body. An opening 22 in the movement direction B is arranged on one side of the receptacle 20. A connection element 50 for connecting the effector body, the tensioning body 40 and the receptacle 20 extends through the opening 22. Alternatively, the receptacle may also have a substantially rectangular cross section, wherein the outer walls of the body joint stabilizing apparatus 1 may have curves for an adaptation to the body geometry of the user.

The tensioning body 40 is arranged outside of the receptacle 20. Here, the tensioning body 40 may be arranged in one body region of the user, wherein the receptacle 20 may be arranged on a different body region of the user. Accordingly, the body joint stabilizing apparatus 1 is able to adaptively damp movements between these two body regions.

A deflection means 70 in the form of a deflection pulley is arranged on the tensioning body 40. The connection element 50 extends over the deflection means. The deflection means 70 deflects the connection element 50 in such a way that the connection element 50 has two portions, both of which extend substantially in the direction of the main movement direction B.

A second end 54 of the connection element 50 is fastened to the outer surface of the receptacle 20, on the same side on which the opening 22 is arranged as well. The connection element 20 has a fibrous embodiment, i.e., an embodiment in the form of a rope. Alternatively, the connection element may also be embodied in the form of a belt, strap, cable and/or wire.

The receptacle 20, the tensioning body 40 and the deflection means 70 are manufactured from polypropylene (PP). Alternatively, the receptacle and the tensioning body could also be manufactured from a different plastic or a metal, such as aluminum, for example, or ceramics. FIG. 1C is a sectional view of the body joint stabilizing apparatus 1 along the sectional line A-A of FIG. 1B. The interior 24 of the receptacle 20, in particular, can be gathered from FIG. 1C. The interior 24 is filled with a shear-thickening medium 30. The shear-thickening medium shown in FIG. 1C is a dilatant fluid. Alternatively, use can also be made of a shear-thickening solid. Moreover, sand can also be used as a medium.

Moreover, an effector body 60 is arranged in the interior 24 of the receptacle 20. The effector body 60 is fastened to the first end 52 of the connection element 50 and can be displaced in the interior 24 in the movement direction B by way of a relative movement between the receptacle 20 and the tensioning body 40, in particular by way of a movement in which the receptacle 20 and the tensioning body 40 move apart. The shear-thickening medium 30 flows around the effector body 60 if the effector body 60 is pulled in the direction of the opening 22 by the connection element 50. The effector body 60 has a shear surface 62, which forms the surface of the effector body 60, in relation to which the shear-thickening medium flows.

If the effector body 60 moves through the shear-thickening medium 30 below a critical speed, the effector body 60 can displace the shear-thickening medium 30 and move in unimpeded fashion to the opening 22. If the tensioning body 40 and the receptacle 20 are moved apart so quickly, i.e., in jerking fashion, that the effector body 60 moves through the shear-thickening medium 30 with a speed greater than or equal to a critical speed, the shear-thickening medium starts to solidify in the region of the shear surfaces 62 of the effector body 60. As a result, the mobility of the effector body 60 in the direction of the receptacle 22 is restricted or prevented, depending on the speed with which the effector body 60 moves.

The configuration of the connection element 50 and the deflection means 70 facilitates a gearing of the movement path, from the tension body 40 to the effector body 60. The body joint stabilizing apparatus 1 shown in FIGS. 1A, 1B and 1C can facilitate a transmission ratio of the path and the speed of 1:2 between the tensioning body 40 and the effector body 60. That is to say, if the tensioning body 40 is spaced apart on account of a movement of the two body regions of the user away from one another by a distance X, then the effector body 60 travels along a path that is twice as long, i.e., 2X, relative to the receptacle 20. The same applies to the speed; if the tensioning body 40 is moved away from the receptacle 20 at the speed V, the effector body 60 moves with twice the speed, i.e., 2V, in relation to the receptacle 20.

In relation to the balance of forces, tensioning body 40 and effector body 60 behave in the ratio of 2:1. That is to say, if a force F acts on the tensioning body 40 when spacing apart the body regions of the user, only half of the force F, i.e. ½ F, acts on the effector body 60.

Accordingly, the deflection means 70 serves as a force converter, by means of which the field of use of the body joint stabilizing apparatus 1 and the dimensioning of the components of the body joint stabilization apparatus 1 can be influenced. By way of example, increasing the path and/or the speed, with which the effector body 60 moves in relation to the receptacle 20, may be helpful for fields of application in which the body regions of the user to be stabilized only have comparatively small and/or slow movements, for which it is difficult to optimally exploit the effect of sheer thickening in the receptacle 20.

The effector body is made of polypropylene (PP). Alternatively, the effector body may also be manufactured from a different plastic or a metal, such as aluminum, for example.

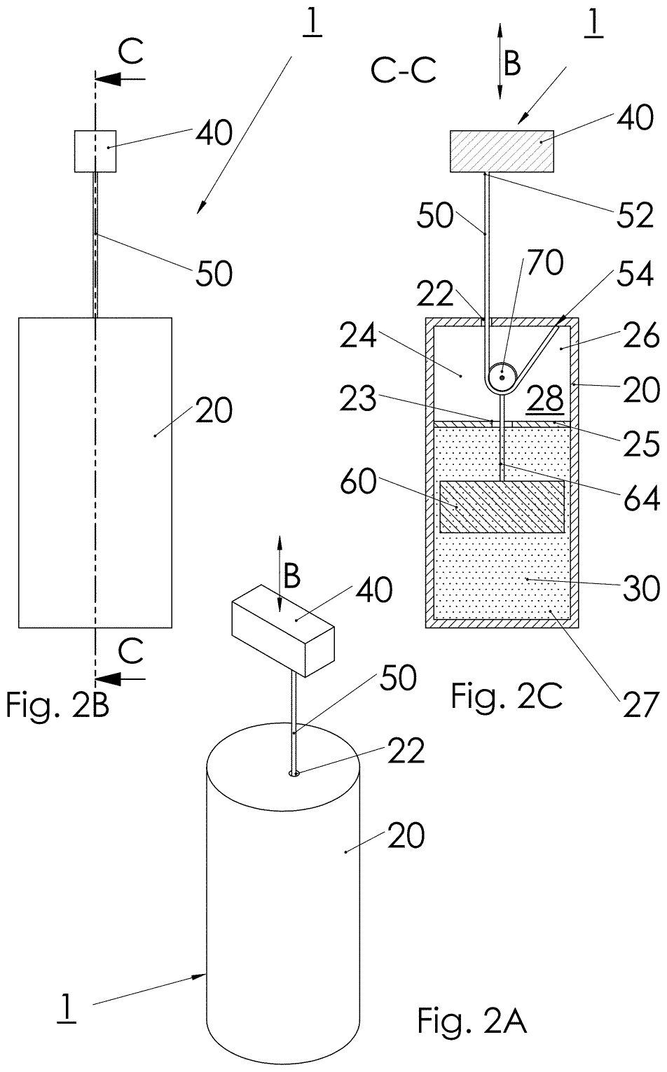

FIGS. 2A, 2B and 2C show a perspective view of a body joint stabilizing apparatus 1 for adaptively dampening a body movement. The body joint stabilizing apparatus 1 of FIGS. 2A, 2B and 2C differs from the body joint stabilizing apparatus of FIGS. 1A, 1B and 1C in that only a portion of the connection element 50 and the tensioning body 40 are arranged outside of the receptacle 20.

FIG. 2C shows a sectional view of the body joint stabilizing apparatus 1 along the section line C-C of FIG. 2B, from where it is possible to gather that the interior 24 of the receptacle 20 is divided into a first chamber 27 and a second chamber 28 by means of a separating wall 25. The effector body 60 is arranged in the first chamber 27. A deflection means 70 in the form of a deflection pulley is arranged in the second chamber 28, said deflection pulley being connected to the effector body 60 by way of a rod element 64. Here, the deflection pulley is rotatably mounted on the rod element 64. The rod element extends from the first chamber 27 into the second chamber 28 through an opening 23 in the separating wall 25. Movements acting on the deflection pulley that emanate from the connection element 50 are transmitted onto the effector body by means of the rod element 64. On account of the arrangement described above, the deflection means is prevented from coming into contact with the shear-thickening medium.

Moreover, two portions of the connection element 50 are arranged in the interior 24 of the receptacle, with one portion extending from the deflection means 70 to the opening 22 and the other portion extending from the deflection means 70 toward the inner wall 26 of the receptacle 20 in the surroundings of the opening 22. Here, both portions of the connection element 50 extend substantially in the movement direction B.

The body joint stabilizing apparatus 1 shown in FIGS. 2A, 2B and 2C has the opposite behavior in relation to the body joint stabilizing apparatus shown in FIGS. 1A, 1B and 1C. The transmission ratio of the tensioning body 40 shown in FIGS. 2A, 2B and 2C in relation to the effector body 60 is 2:1 in relation to the path and the speed. Thus, when the tensioning body 40 and the receptacle 20 move apart, the tensioning body 40 travels twice the path length in relation to the effector body 60.

The same applies to the ratio the speeds with which the tensioning body 40 and effector body 60 move in the case of a deflection of the body joint stabilizing apparatus 1. That is to say, the tensioning body 40 moves with twice the speed in relation to the effector body 60 when the tensioning body 40 and the receptacle 20 move apart. The force ratio has the opposite behavior. Thus, a force that is half the size of the force acting on the effector body 60 acts on the pullout body 40 in the case of a deflection of the body joint stabilizing apparatus 1, i.e., when the tensioning body 40 and the receptacle 20 move apart.

By way of example, if the receptacle 20 in the tensioning body 40 move apart in such a way that a force of 500 N acts on the tensioning body 40, a force of 1000 N acts on the effector body 60.

Such a configuration of the deflection means 70 and the connection element 50 can be helpful for fields of application in which there are comparatively large and/or fast movements between the body regions to be stabilized. Thus, the reduction in the scope of movement of the effector body 60 allows a smaller size of the body joint stabilizing apparatus 1.

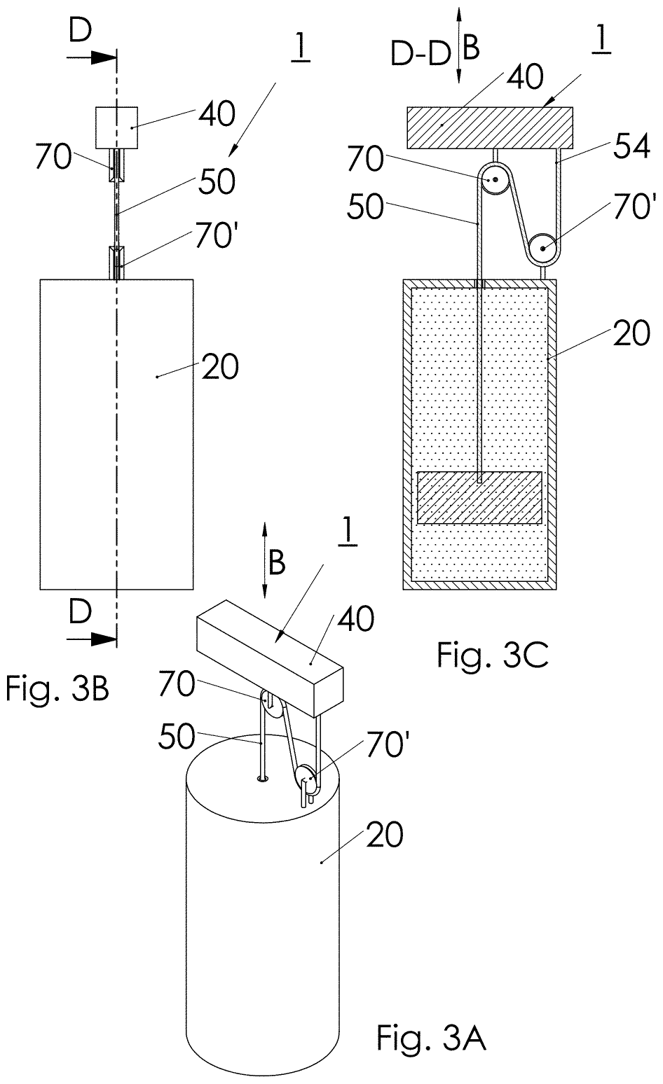

FIGS. 3A, 3B and 3C show a body joint stabilizing apparatus 1 for adaptively damping a body movement. The body joint stabilizing apparatus 1 shown in FIGS. 3A, 3B and 3C differs from the body joint stabilizing apparatus shown in FIGS. 1A, 1B and 1C in that a further deflection means 70′ is arranged on the receptacle 20, said further deflection means additionally deflecting the connection element. Accordingly, the connection element 50 has a third portion, which extends virtually in the movement direction B from the deflection pulley 70′ arranged on the receptacle 20 to the tensioning body 40. A second end 54 of the connection element 50 is fastened to the tensioning body 40.

In relation to the tensile force, the body joint stabilizing apparatus 1 shown in FIGS. 3A, 3B and 3C has a transmission ratio of 3 to 1 between the tensioning body 40 and the effector body 60. The transmission ratio between the effector body 60 and the receptacle 20 is 1 to 2 in relation to the tensile force.

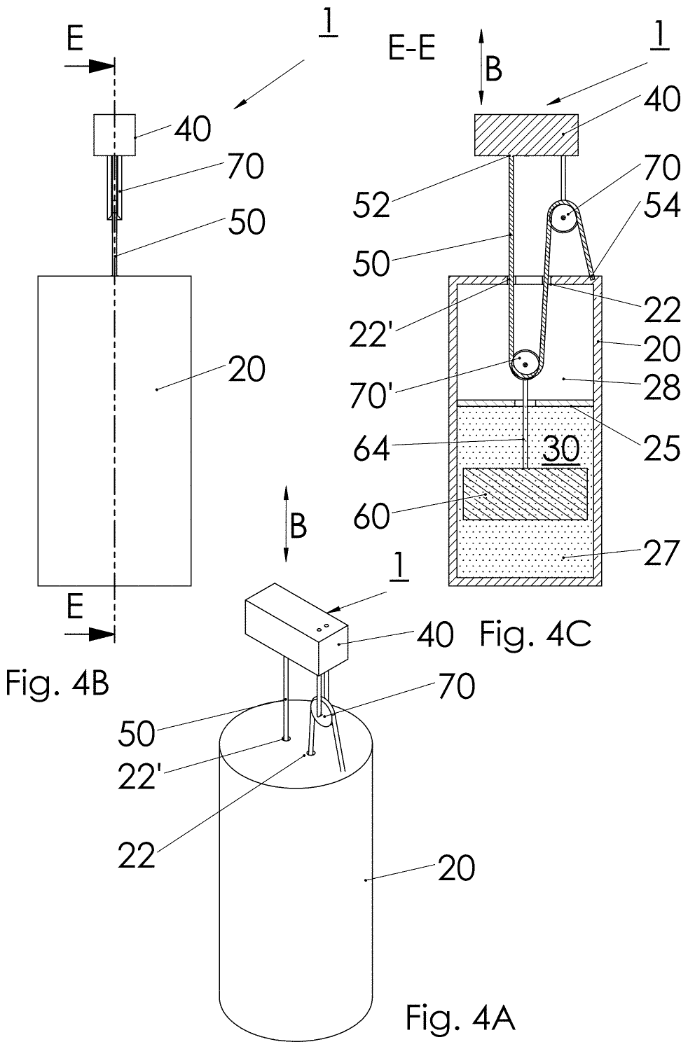

FIGS. 4A, 4B and 4C show a body joint stabilizing apparatus 1 for adaptively damping a body movement. The body joint stabilizing apparatus 1 shown in FIGS. 4A, 4B and 4C differs from the body joint stabilizing apparatus shown in FIGS. 1A, 1B and 1C in that a further deflection means 70′ is arranged on the effector body 60, said further deflection means additionally deflecting the connection element 50. Accordingly, the connection element 50 has a third portion, which extends virtually along the movement direction B and which emerges from the receptacle 20 through a further opening 22′. The first end 52 of the connection element 50 is fastened to the tensioning body 40.

It can further be gathered from FIG. 4C that the receptacle 20, as shown in FIG. 2C, is divided into a first chamber 27 and a second chamber 28 by means of a separating wall 25 in order to is prevent the deflection means 70′ from coming into contact with the shear-thickening medium 30. Here, the deflection means 70′ is connected to the effector body 60 via a rod element 64.

In relation to the tensile force, the body joint stabilizing apparatus 1 shown in FIGS. 4A, 4B and 4C has a transmission ratio of 3 to 2 between the tensioning body 40 and the effector body 60. The transmission ratio between the effector body 60 and the receptacle 20 is 2 to 1 in relation to the tensile force.

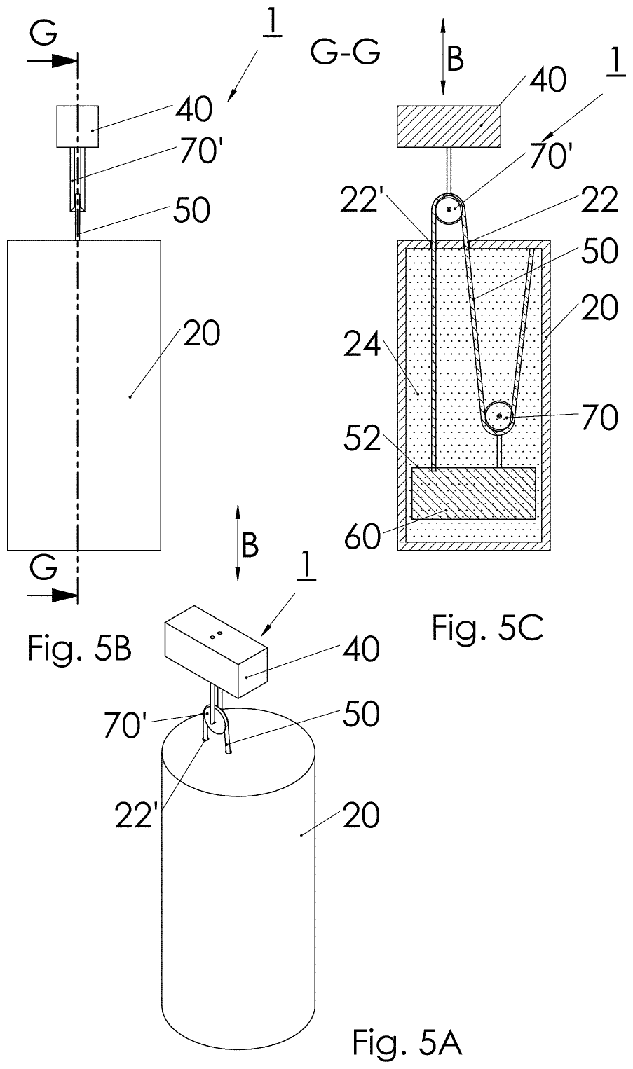

FIGS. 5A, 5B and 5C show a body joint stabilizing apparatus 1 for adaptively damping a body movement. The body joint stabilizing apparatus 1 shown in FIGS. 5A, 5B and 5C differs from the body joint stabilizing apparatus shown in FIGS. 2A, 2B and 2C in that a further deflection means 70′ is arranged on the tensioning body 40, said further deflection means additionally deflecting the connection element 50. Accordingly, the connection element 50 has a third portion, which extends virtually along the movement direction B and which enters the receptacle 20 through a further opening 22′. The first end 52 of the connection element 50 is fastened to the effector body 60. Moreover, the interior 24 of the receptacle 20 is not divided by a separating wall.

In relation to the tensile force, the body joint stabilizing apparatus 1 shown in FIGS. 5A, 5B and 5C has a transmission ratio of 2 to 3 between the tensioning body 40 and the effector body 60. The transmission ratio between the effector body 60 and the receptacle 20 is 3 to 1 in relation to the tensile force.

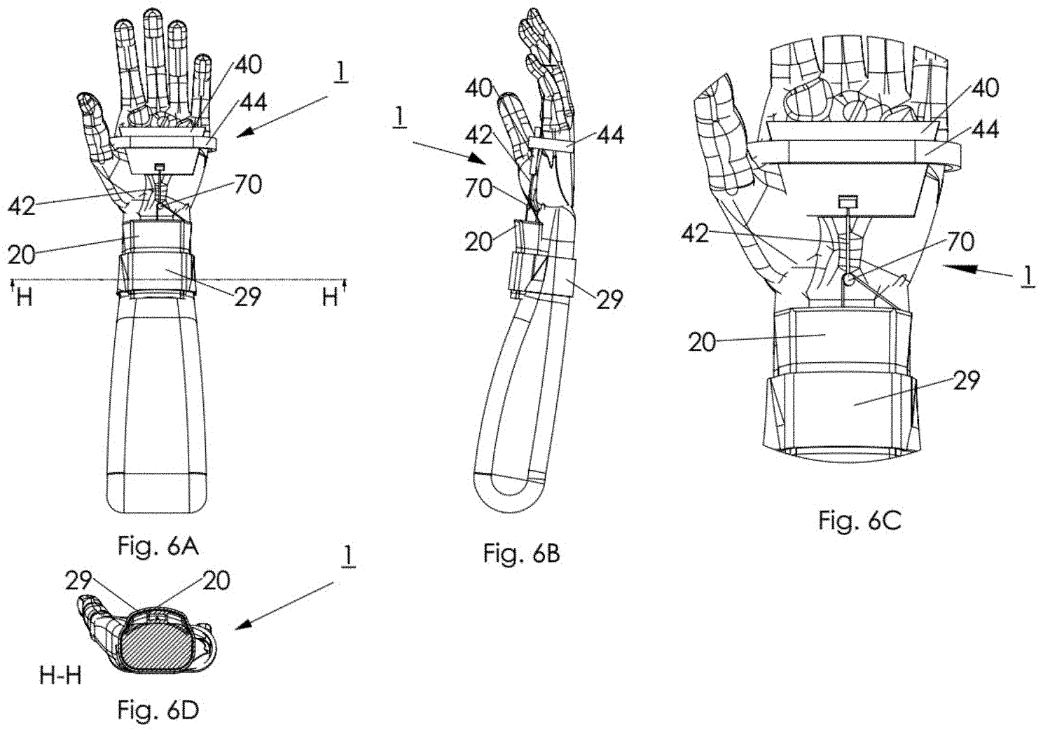

FIGS. 6A, 6B, 6C and 6D show a body joint stabilizing apparatus 1 for adaptively damping a body movement, which is attached to a hand and a forearm for stabilizing the wrist. The body joint stabilizing apparatus 1 shown in FIGS. 6A, 6B and 6C exhibits fastening loops 29, 44. The fastening loop 29 serves to fasten the receptacle 20 to the forearm. The fastening loop 44 serves to fasten the tensioning body 40 to the hand.

The fastening loops 29, 44 are securely connected to the receptacle 20 and the tensioning body 40, respectively, and adhere to the receptacle 20 and the tensioning body 40, respectively, by way of an adhesive bond, for example. Alternatively, the fastening loops may also have an integral embodiment with the receptacle and the tensioning body, respectively. In a further alternative, the fastening loops hold the receptacle and the tensioning body by way of the principle of static friction.

Here, the fastening loops can be closed by means of a hook-and-loop fastener. Alternatively, the fastening loops could also consist of closed elastic bands, with the loops, by way of their elastic properties, ensuring that the receptacle and the tensioning body are held against the body of the user.

In a further alternative, the fastening loop for fastening the tensioning body may also be replaced by a glove, wherein the tensioning body is integrated in the glove and securely connected to the latter.

In a yet further alternative, the fastening loops can be replaced by a bandage or tape, which are wound around the hand and the forearm, respectively, in order to fasten the tensioning body and the receptacle, respectively.

The body joint stabilizing apparatus 1 shown in FIG. 6A, 6B and 6C further shows that the tensioning body 40 comprises a rod element 42, on which a deflection means 70 is fastened. Moreover, it is possible to gather from FIGS. 6A, 6B, 6C and 6D that the receptacle 20 is adapted to the form of the forearm.

The functional principle of the body joint stabilizing apparatus 1 shown in 6A, 6B, 6C and 6D corresponds to that of the body joint stabilizing apparatus shown in FIGS. 1A, 1B and 1C. Accordingly, the body joint stabilizing apparatus 1 is able to adaptively dampen movements between the hand and the forearm, i.e., movements of the wrist.

FIG. 6D is a sectional view of the body joint stabilizing apparatus 1 along the section line H-H of FIG. 6A. FIG. 6D shows the fastening loop 29, which completely runs around the forearm and thus fastens the receptacle 20 to the forearm.

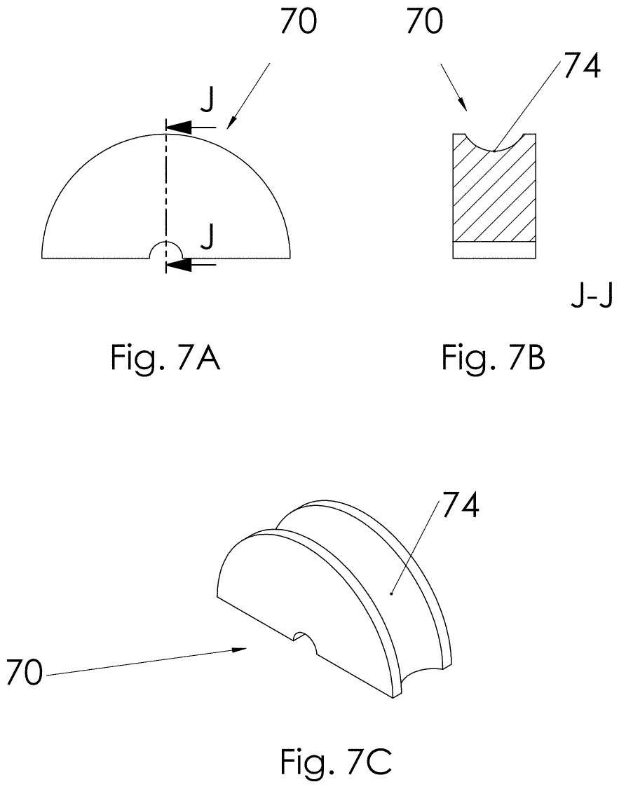

FIG. 7C shows a perspective detailed view of a deflection means 70 in the form of a friction wedge. The friction wedge is able to deflect a connection element through approximately 180°. In order to receive and guide the connection element, the friction wedge has a race 74. FIG. 7A shows a side view of the friction wedge. FIG. 7B shows a sectional view of the friction wedge along the section line J-J of FIG. 7A.

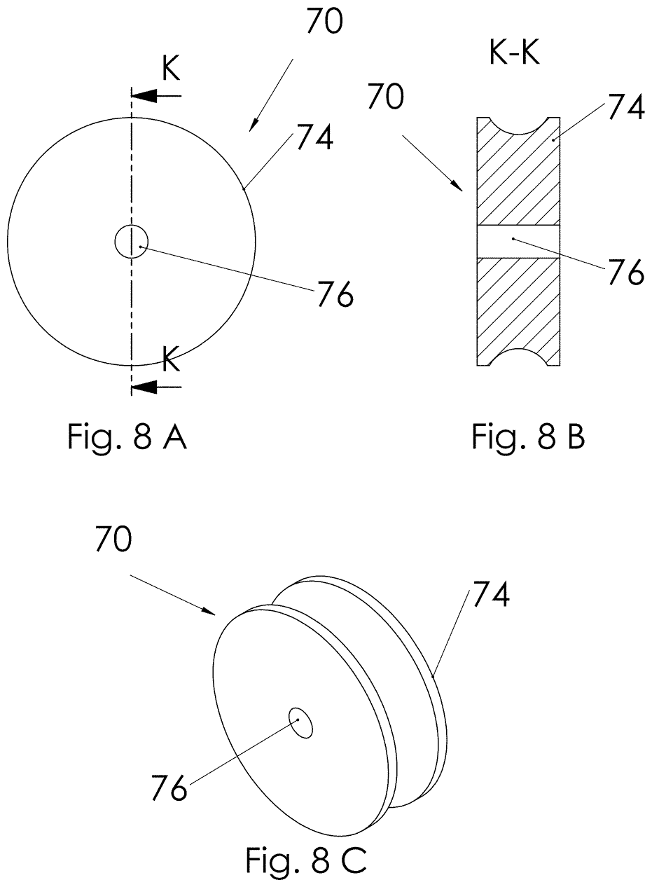

FIG. 8C shows a deflection means 70 in the form of a deflection pulley. The deflection pulley has a race 74 for receiving and guiding a connection. Moreover, the deflection pulley has a bearing 76 for rotatably bearing the deflection pulley. By way of example, the bearing 76 can be embodied in the form of a ball bearing. FIG. 8A shows a side view of the deflection pulley. FIG. 8B shows a sectional view of the deflection pulley along the section line K-K of FIG. 7A.

That deflection means shown in FIGS. 7A to 8C are manufactured from polypropylene (PP). Alternatively, the friction wedge or the deflection pulley may also be manufactured from a different plastic. Moreover, the friction wedge or the deflection pulley may also be manufactured from metals such as aluminum, magnesium, steel, for example, or other low-friction materials.

Return means may be provided in order to bring the body joint stabilizing apparatuses illustrated in the preceding figures back into an initial position. These return means could have an elastic embodiment, for example, and connect the effector body to the side of the receptacle lying opposite the opening. If the effector body is deflected from the initial position by the action of a force, the elastic return means is stretched. Once the external force and the holding force of the shear thickening abate, the elastic return means can convey the effector body, the connection element and the tensioning body back into the initial position on account of the previously experienced stretch.

As far as this is applicable, all individual features that are illustrated in the individual exemplary embodiments can be combined with one another and/or can replace one another, without departing from the scope of the invention.

### LIST OF REFERENCE SIGNS

1 Body joint stabilizing apparatus

### 20 Receptacle

### 22,22′ Opening

### 23 Opening

### 24 Interior

25 Separating wall
26 Inner wall
27 First chamber
28 Second chamber
29 Fastening loop
30 Shear-thickening medium
40 Tensioning body
42 Rod element
44 Fastening loop
50 Connection element

### 52 First end

### 54 Second end

60 Effector body
62 Shear surface
64 Rod element
70, 70′ Deflection means

### 74 Race

### 76 Bearing

B Movement direction

1. A body joint stabilizing apparatus for adaptively damping a body movement, comprising:
a receptacle, wherein the receptacle is filled with a shear-thickening medium,
a tensioning body that is movable relative to the receptacle, wherein the tensioning body is connectable to a body region of the user that is movable relative to another body region of the user, on which the receptacle is fastenable,
an effector body that is displaceably arranged in the receptacle and provided for an interaction with the shear-thickening medium, and
a connection element for transmitting forces between the receptacle and the tensioning body,
wherein the receptacle, the tensioning body and the effector body are connected to one another by way of the connection element, wherein the connection element is guided via at least one deflection means for the purposes of dividing the force acting on the connection element.

2. The body joint stabilizing apparatus of claim 1, wherein the connection element comprises a first end and a second end, and wherein the at least one deflection means is guided in movable fashion on the connection element between the first end and the second end.

3. The body joint stabilizing apparatus of claim 2, wherein the first end of the connection element is connected to the effector body or the tensioning body and the second end of the connection element is connected to the receptacle or the tensioning body.

4. The body joint stabilizing apparatus of claim 2, wherein the deflection means deflects the connection element at least once through an angle of between 150° and 190°.

5. The body joint stabilizing apparatus of claim 2, wherein the deflection means deflects the connection element at least once through substantially 180°.

6. The body joint stabilizing apparatus of claim 1, wherein the tensioning body is arranged outside of the receptacle (20).

7. The body joint stabilizing apparatus of claim 2, wherein the deflection means comprises a material with a low coefficient of friction.

8. The body joint stabilizing apparatus of claim 2 wherein the deflection means comprises at least one deflection pulley.

9. The body joint stabilizing apparatus of claim 1 the connection element has is tension-resistant.

10. The body joint stabilizing apparatus of claim 1 wherein the connection element is embodied in the form of an elongate, flexible tension element.

11. The body joint stabilizing apparatus of claim 1 wherein the connection element comprises a connection element selected from the group consisting of fibers, a strap, a belt, a rope, a cable and a wire.

12. The body joint stabilizing apparatus of claim 1 wherein the connection element is integral with the effector body (60).

13. The body joint stabilizing apparatus of claim 2 wherein the deflection means comprises at least two deflection pulleys for deflecting the connection element, with one deflection pulley being arranged on the tensioning body and the other deflection pulley being arranged on the effector body or on the receptacle.

14. The body joint stabilizing apparatus of claim 1 wherein the the interaction between the effector body and the shear-thickening medium are configured in such a way that the effector body is movable through the shear-thickening medium if a force acts on the effector body with a speed below a threshold and that the shear-thickening medium inhibits the movement of the effector body if a force acts on the effector body with a speed that is greater than or equal to a threshold.

15. The body joint stabilizing apparatus of claim 1 wherein the shear-thickening medium comprises a solid, a polymer, a powder, a fluid, a paste, a gel, or any combination thereof.

16. The body joint stabilizing apparatus of claim 1 wherein the receptacle comprises an integral embodiment with a frame for fastening the body joint stabilizing apparatus to a body part.
